(19) United States
(12) Patent Application Publication
(10) Pub. No.: US 2021/0232705 A1
(43) Pub. Date: Jul. 29, 2021

(54) METHOD AND SYSTEM FOR GENERATING SYNTHETICALLY ANONYMIZED DATA FOR A GIVEN TASK

(71) Applicant: IMAGIA CYBERNETICS INC., Montréal (CA)

(72) Inventors: Florent CHANDELIER, Lery (CA); Andrew JESSON, Outremont (CA); Mohammad HAVAEI, Montréal (CA); Lisa DIJORIO, Montréal (CA); Cevile LOW-KAM, Montréal (CA); Nicolas CHAPADOS, Montréal (CA)

(73) Assignee: IMAGIA CYBERNETICS INC., Montréal, QC (CA)

(21) Appl. No.: 17/259,908

(22) PCT Filed: Jul. 12, 2019
(86) PCT No.: PCT/IB2019/055972

§ 371 Date: Jan. 12, 2021

Related U.S. Application Data
(60) Provisional application No. 62/697,804, filed on Jul. 13, 2018.

(51) Int. Cl. G06F 21/62; G06N 20/00; G16H 10/60

(52) U.S. Cl. CPC G06F 21/6254; G16H 10/60; G06N 20/00

(57) ABSTRACT

A method and a system are disclosed for generating synthetically anonymized data, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data and the first data; providing a task-specific embedding comprising task-specific features, wherein said task-specific features enables a disentanglement of different classes relevant to the given task; generating synthetically anonymized data, the generating comprising a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process.

### TECHNICAL FIELD

The invention relates to data processing. More precisely, the invention pertains to a method and system for generating synthetically anonymized data for a given task.

### BACKGROUND

Being able to provide anonymized data is of great interest for various reasons.

Recently, AI methods have been introduced as part of the Statistical methods protecting sensitive information or the identity of the data owner have become critical to ensure privacy of individuals as well as of organizations.

Specifically, sharing individual-level data from clinical studies remains challenging. The status quo often requires scientists to establish a formal collaboration and execute extensive data usage agreements before sharing data. These requirements slow or even prevent data sharing between researchers in all but the closest collaborations and are serious drawbacks.

Recent initiatives have begun to address cultural challenges around data sharing. In recent years, many datasets containing sensitive information about individuals have been released into public domain with the goal of facilitating data mining research.

Databases are frequently anonymized by simply suppressing identifiers that reveal the identities of the users, like names or identity numbers.

Different processes (https://arxiv.org/pdf/1802.09386.pdf; https://arxiv.org/pdf/1803.11556.pdf; https://www.biorxiv.org/content/biorxiv/early/2017/07/05/159756.full.pdf; https://openreview.net/forum?id=rJv4XWZA-) are of great value in the anonymization process of data to either augment training data (See Synthetic data augmentation using GAN for improved liver lesion classification http://www.eng.biu.ac.il/goldbej/files/2018/01/ISBI_2018_Maayan.pdf) or share subject data, however they do not feature the following two requirements: (1) a guarantee that the generated data is not identifiable (background attacks, including attacks if you know, a posteriori, tasks for which the anonymized data was well suited for), and (2) a guarantee that the generated data is relevant for a subsequent task (disentangling appropriate factors of task-specific variations).

There is a need for a method and system that will overcome at least one of the above-identified drawbacks.

Features of the invention will be apparent from review of the disclosure, drawings and description of the invention below.

### BRIEF SUMMARY

According to a broad aspect, there is disclosed a method for generating synthetically anonymized data for a given task, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data and the first data; providing a task-specific embedding comprising task-specific features suitable for said task, wherein said task-specific features enable a disentanglement of different classes relevant to the given task; generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and providing the generated synthetically anonymized data for the given task.

In accordance with an embodiment, the generating of the synthetically anonymized data for the given task comprises checking that the synthetically anonymized data is dissimilar to the first data to be anonymized for a given metric and the generated synthetically anonymized data for the given task is provided if said checking is successful.

According to an embodiment, the first data comprises patient data.

According to an embodiment, the providing of the task-specific embedding comprising task specific features suitable for said task comprises obtaining an indication of the given task; obtaining an indication of classes relevant to the given task; obtaining a model suitable for performing a disentanglement of the data for the given task; and generating the task-specific embedding using the obtained model, the indication of classes relevant to the given task, the indication of the given task and the data.

According to an embodiment, the providing of the identifier embedding comprising identifiable features comprises obtaining data used for identifying the identifiable features; obtaining a model suitable for identifying the identifiable features in said data; obtaining an indication of identifiable entities and generating the identifier embedding using the model suitable for identifying the identifiable features, the indication of identifiable entities and the data to be used for identifying the identifiable features.

According to an embodiment, the data comprises the data used for identifying the identifiable features.

According to an embodiment, the model suitable for identifying the identifiable features in the data comprises a Single Shot MultiBox Detector (SSD) model.

According to an embodiment, the model suitable for performing a disentanglement of the data for the given task comprises one of an Adversarially Learned Mixture Model (AMM) in one of a supervised, semi supervised or unsupervised training.

According to an embodiment, the indication of identifiable entities comprises one of a number of classes and an indication of a class corresponding to at least one of said data.

According to an embodiment, the indication of identifiable entities comprises at least one box locating at least one corresponding identifiable entity.

According to a broad aspect, there is disclosed a non-transitory computer readable storage medium for storing computer-executable instructions which, when executed, cause a computer to perform a method for generating synthetically anonymized data for a given task, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data and the first data; providing a task-specific embedding comprising task-specific features suitable for said task, wherein said task-specific features enables a disentanglement of different classes relevant to the given task; generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and providing the generated synthetically anonymized data for the given task.

According to another broad aspect, there is disclosed a computer comprising a central processing unit; a display device; a communication unit; a memory unit comprising an application for generating synthetically anonymized data for a given task, the application comprising instructions for providing first data to be anonymized; instructions for providing a data embedding comprising data features, wherein data features enable a representation of corresponding data, and wherein the data is representative of the first data; instructions for providing an identifier embedding comprising identifiable features, wherein the identifiable features enable an identification of the data and the first data; instructions for providing a task-specific embedding comprising task-specific features suitable for said task, wherein said task-specific features enables a disentanglement of different classes relevant to the given task; instructions for generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and instructions for providing the generated synthetically anonymized data for the given task.

It is an object to provide a method and a system which by design ensure anonymization of data based on an amendment of a defined set of identifiable features in data to prevent a re-identifying of the data.

It is another object to provide a method and a system which by design ensure that synthetic anonymized data conveys a suitable representation for processing the anonymized data for a given task.

The method disclosed herein is of great advantage for various reasons.

In fact, a first advantage of the method disclosed is that it provides privacy by-design for an anonymization process, while ensuring that the anonymized data is relevant for further research pertaining to a given task and to be representative of the general “look'n'feel” of the original data.

A second advantage of the method disclosed herein is that it enables the sharing of patient data in an open innovation environment, while ensuring patient privacy and control over the specific characteristics of the anonymized data (representative of all patient or sub-population thereof, representative globally of a task or sub-classes thereof).

A third advantage of the method disclosed herein is that it provides ways to anonymize data without an a-priori on what aspects of the data may convey such privacy risk(s); accordingly as such risk evolves, the method disclosed herein may adapt and benefit from further research and development in the field of data privacy.

### BRIEF DESCRIPTION OF THE DRAWINGS

In order that the invention may be readily understood, embodiments of the invention are illustrated by way of example in the accompanying drawings.

FIG. 1 is a flowchart which shows an embodiment of a method for generating synthetically anonymized data for a given task. The method comprises inter alia, providing a task-specific embedding comprising task-specific features. The method further comprises providing an identifier embedding comprising identifiable features.

FIG. 2 is a flowchart which shows an embodiment for providing an identifier embedding comprising identifiable features.

FIG. 3 is a flowchart which shows an embodiment for providing the task-specific embedding comprising task-specific features.

FIG. 4 is a diagram which shows an embodiment of a system for generating synthetically anonymized data for a given task.

FIG. 5 is a diagram which shows an embodiment of an Adversarially Learned Mixture Model (AMM) which may be used in an embodiment of the method for generating synthetically anonymized data for a given task.

Further details of the invention and its advantages will be apparent from the detailed description included below.

### DETAILED DESCRIPTION

In the following description of the embodiments, references to the accompanying drawings are by way of illustration of an example by which the invention may be practiced.

### Terms

The term “invention” and the like mean “the one or more inventions disclosed in this application,” unless expressly specified otherwise.

The terms “an aspect,” “an embodiment,” “embodiment,” “embodiments,” “the embodiment,” “the embodiments,” “one or more embodiments,” “some embodiments,” “certain embodiments,” “one embodiment,” “another embodiment” and the like mean “one or more (but not all) embodiments of the disclosed invention(s),” unless expressly specified otherwise.

A reference to “another embodiment” or “another aspect” in describing an embodiment does not imply that the referenced embodiment is mutually exclusive with another embodiment (e.g., an embodiment described before the referenced embodiment), unless expressly specified otherwise.

The terms “including,” “comprising” and variations thereof mean “including but not limited to,” unless expressly specified otherwise.

The terms “a,” “an” and “the” mean “one or more,” unless expressly specified otherwise.

The term “plurality” means “two or more,” unless expressly specified otherwise.

The term “herein” means “in the present application, including anything which may be incorporated by reference,” unless expressly specified otherwise.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “e.g.” and like terms mean “for example,” and thus do not limit the terms or phrases they explain.

The term “i.e.” and like terms mean “that is,” and thus limit the terms or phrases they explain.

The term “disentanglement” and like terms means in the real world that a models seek to represent, there are some factors of variation that can be modified independently, and others that cannot be (or, for practical purposes, never are). A trivial example of this is: if you're modeling pictures of people, then someone's clothing is independent of their height, whereas the length of their left leg is strongly dependent on the length of their right leg. The goal of disentangled features can be most easily understood as wanting to use each dimension of a latent z code to encode one and only one of these underlying independent factors of variation. Using the example from above, a disentangled representation would represent someone's height and clothing as separate dimensions of the z code.

The term “embedding” and like terms means relatively low-dimensional space into which high-dimensional vectors (dimensionality reduction) can be translated into. Embeddings make it easier to do machine learning on large inputs such as sparse vectors representing words or image characteristics. Ideally, an embedding captures some of the semantics of the input by placing semantically similar inputs close together (contextual similarity) in the embedding space. It will be appreciated that an embedding can be learned and reused across models. The purpose of an embedding is to map any input object (e.g. word, image) into vectors of real numbers, which algorithms, like deep learning, can then ingest and process, to formulate an understanding. The individual dimensions in these vectors typically have no inherent meaning. Instead, it is the overall patterns of location and distance between vectors that machine learning takes advantage of.

The term “feature” and like terms means, in machine learning and pattern recognition, an individual measurable property or characteristic of a phenomenon being observed. The concept of “feature” is related to that of explanatory variable used in statistical techniques such as linear regression. A feature vector is an n-dimensional vector of numerical features that represent some object. The vector space associated with these vectors is often called the feature space. In machine learning, feature learning or representation learning is a set of techniques that enables a system to automatically discover the representations needed for feature detection or classification from raw data. This replaces manual feature engineering and allows a machine to both learn the features and use them to perform a specific task. A classifier or neural network needs to be trained to learn to extract features from data. The features learned by a neural network depend among other things on the cost function used during training. The cost function defines the task that has to be solved. In order to have the ability to classify, the network is trained to minimize the classification error over training points. The embedding encodes the features extracted from the data. Multilayer neural networks can be used to perform feature learning, since they learn a representation of their input at the hidden layer(s) which is subsequently used for classification or regression at the output layer. Deep neural networks learn feature embeddings of the input data that enable state-of-the-art performance in a wide range of computer vision tasks.

The term “generative” and like terms means a way of learning any kind of data distribution using unsupervised learning and it has achieved tremendous success in just a few years. All types of generative models aim at learning the true data distribution of the training set so as to generate new data points with some variations. But it is not always possible to learn the exact distribution of the data either implicitly or explicitly and so we try to model a distribution which is as similar as possible to the true data distribution. Two of the most commonly used and efficient approaches are Variational Autoencoders (VAE) and Generative Adversarial Networks (GAN). Variational Autoencoders (VAE) aim at maximizing the lower bound of the data log-likelihood and Generative Adversarial Networks (GAN) aim at achieving an equilibrium between generator and discriminator.

Sampling—in Generative modeling with sampling can be considered one of the hardest tasks, it implies the ability to generate data that resemble the data used during training in the sense that they should ideally follow the same, unknown, true distribution. If data x are generated from an unknown distribution p such that x□p(x) p can be approximated by learning a distribution q, from which it is possible to efficiently sample, that is close enough to p. This task is intimately related to probabilistic modeling and probability density estimation, but the focus is on the ability to generate good samples efficiently, rather than obtaining a precise numerical estimation of the probability density at a given point. There is a direct relation between “Generative” since sampling can generate synthetic data points.

Neither the Title nor the Abstract is to be taken as limiting in any way as the scope of the disclosed invention(s). The title of the present application and headings of sections provided in the present application are for convenience only, and are not to be taken as limiting the disclosure in any way.

Numerous embodiments are described in the present application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural and logical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

With all this in mind, the present invention is directed to a method and a system for generating synthetically anonymized data for a given task.

It will be appreciated that the method may be used in various embodiments. For instance in the medical field, the method may be used for generating synthetically anonymized patient data.

It will be appreciated that the given task to perform may be of various types.

In fact, the given task to perform is defined as any task in which the data may be used to.

For instance, in the medical field, the given task to perform may be used in one embodiment to determine an outcome of a patient in response to a treatment. In one embodiment, the given task to perform may be to provide a diagnostic. In another embodiment, the given task to perform may be one of anomaly detection and location (e.g. on images, on 1-D longitudinal information such as EKG), precision medicine prediction from various input information (e.g. images, clinical reports, EHR patient history), treatment strategy clinical decision support, drug side-effect prediction, relapse and metastasis prediction, readmission rate, post-operative surgical complication, assisted surgery and assisted robotic surgery, preventative health prediction (e.g. Alzheimer, Parkinson, cardiac event or depression predictions).

It will be appreciated that the method and the system disclosed are of great advantage for many reasons, as explained further below.

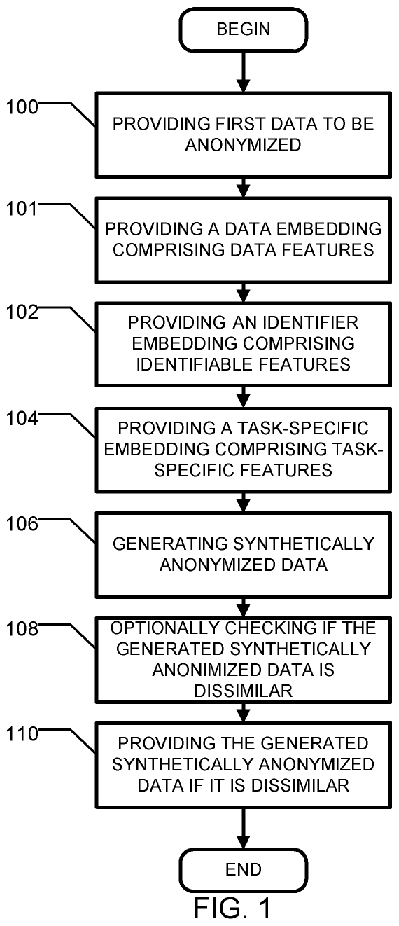

Now referring to FIG. 1, there is shown an embodiment of a method for generating synthetically anonymized data for a given task.

It will be appreciated that the data may be any type of data which may be identified.

For instance and in accordance with an embodiment, the data comprises patient data. The skilled addressee will appreciate that the patient data may be identifiable since it is associated with a given patient.

In another embodiment, the data is one of patient image data (e.g. CT scans, MRI, ultrasound, PET, X-rays), clinical reports, lab and pharmacy reports.

It will be appreciated that the task is a processing to be performed using the data, to further predict downstream aspects related to the data, or classify the data. Generally speaking, a task may refer to one of a regression, a classification, a clustering, a multivariate querying, a density estimation, a dimension reduction and a testing and matching.

It will be appreciated that the method disclosed herein for generating synthetically anonymized data for a given task may be implemented according to various embodiments.

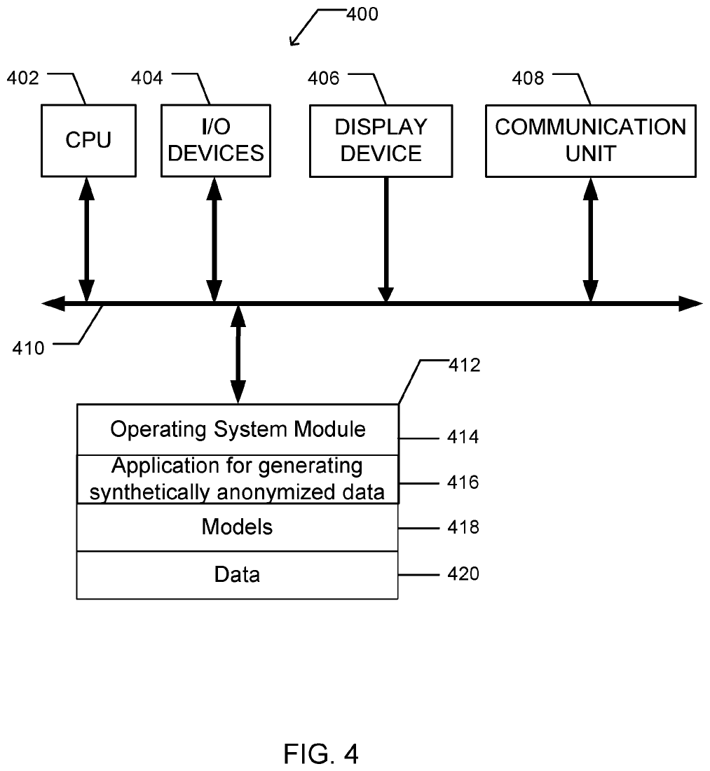

Now referring to FIG. 4, there is shown an embodiment of a system for implementing the method disclosed herein for generating synthetically anonymized data for a given task. In this embodiment, the system comprises a computer 400. It will be appreciated that the computer 400 may be any type of computer.

In one embodiment, the computer 400 is selected from a group consisting of desktop computers, laptop computers, tablet PC's, servers, smartphones, etc. It will also be appreciated that, in the foregoing, the computer 400 may also be broadly referred to as a processor.

In the embodiment shown in FIG. 4, the computer 400 comprises a central processing unit (CPU) 402, also referred to as a microprocessor, input/output devices 404, a display device 406, a communication unit 408, a data bus 410 and a memory unit 412.

The central processing unit 402 is used for processing computer instructions. The skilled addressee will appreciate that various embodiments of the central processing unit 402 may be provided.

In one embodiment, the central processing unit 402 comprises a CPU Core i5 3210 running at 2.5 GHz and manufactured by Intel™.

The input/output devices 404 are used for inputting/outputting data into the computer 400.

The display device 406 is used for displaying data to a user. The skilled addressee will appreciate that various types of display device 406 may be used.

In one embodiment, the display device 406 is a standard liquid crystal display (LCD) monitor.

The communication unit 408 is used for sharing data with the computer 400.

The communication unit 408 may comprise, for instance, universal serial bus (USB) ports for connecting a keyboard and a mouse to the computer 400.

The communication unit 408 may further comprise a data network communication port such as an IEEE 802.3 port for enabling a connection of the computer 400 with a remote processing unit, not shown.

The skilled addressee will appreciate that various alternative embodiments of the communication unit 408 may be provided.

The memory unit 412 is used for storing computer-executable instructions.

The memory unit 412 may comprise a system memory such as a high-speed random access memory (RAM) for storing system control program (e.g., BIOS, operating system module, applications, etc.) and a read-only memory (ROM).

It will be appreciated that the memory unit 412 comprises, in one embodiment, an operating system module 414.

It will be appreciated that the operating system module 414 may be of various types.

In one embodiment, the operating system module 414 is OS X Yosemite manufactured by Apple™. In another embodiment, the operating system module 414 comprises Linux Ubuntu 18.04.

The memory unit 412 further comprises an application for generating synthetically anonymized data 416.

The memory unit 412 further comprises models used by the application for generating synthetically anonymized data 416.

The memory unit 412 further comprises data used by the application for generating synthetically anonymized data 416.

Now referring back to FIG. 1 and according to processing step 100, a first data to be anonymized is provided.

It will be appreciated that the first data to be anonymized may be provided according to various embodiments. In accordance with an embodiment, the first data to be anonymized is obtained from the memory unit 412 of the computer 400.

In accordance with another embodiment, the first data to be anonymized is provided by a user interacting with the computer 400.

In accordance with yet another embodiment, the first data to be anonymized is obtained from a remote processing unit operatively coupled with the computer 400. It will be appreciated that the remote processing unit may be operatively coupled with the computer 400 according to various embodiments. In one embodiment, the remote processing unit is operatively coupled with the computer 400 via a data network selected from a group comprising at least one of a local area network, a metropolitan area network and a wide area network. In one embodiment, the data network comprises the Internet.

As mentioned above, it will be appreciated that in one embodiment the first data to be anonymized comprises patient data.

According to processing step 101, a data embedding comprising data features is provided. It will be appreciated that the data features enable a representation of corresponding data and the data is representative of the first data.

In one embodiment, the data embedding is obtained by training a deep generative model in a representation learning task, onto the data itself, such as disclosed in “representation learning: a review and new perspectives—arXiv:1206.5538”, in “Variational lossy autoencoder. arXiv:1611.02731”, in “neural discrete representation learning—arXiv:1711.00937” and in “Privacy-preserving generative deep neural networks support clinical data sharing—bioarxkiv:159756”.

Moreover, it will be appreciated that the data embedding may be provided according to various embodiments. In accordance with an embodiment, the data embedding is obtained from the memory unit 412 of the computer 400.

In accordance with another embodiment, the data embedding is provided by a user interacting with the computer 400.

In accordance with yet another embodiment, the data embedding is obtained from a remote processing unit operatively coupled with the computer 400.

Still referring to FIG. 1 and according to processing step 102, an identifier embedding comprising identifiable features is provided. It will be appreciated that the identifiable features enable an identification of the data and the first data.

It will be appreciated by the skilled addressee that the identifier embedding comprising identifiable features may be provided according to various embodiments.

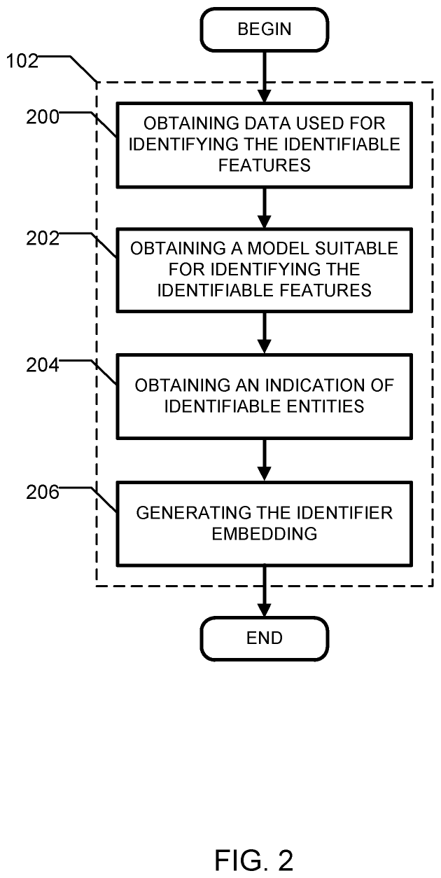

Now referring to FIG. 2, there is shown an embodiment for providing the identifier embedding comprising the identifiable features.

According to processing step 200, data used for identifying the identifiable features is obtained.

It will be appreciated that the data used for identifying features may be of various types. In one embodiment, the data used for identifying the identifiable features comprises at least one portion of the first data provided.

In accordance with another embodiment, the data used for identifying the identifiable features may be different data than the first data provided according to processing step 100.

It will be also appreciated that the data used for identifying the identifiable features may be provided according to various embodiments.

In accordance with an embodiment, the data used for identifying the identifiable features is obtained from the memory unit 412 of the computer 400.

In accordance with another embodiment, the data used for identifying the identifiable features is provided by a user interacting with the computer 400.

In accordance with yet another embodiment, the data used for identifying the identifiable features is obtained from a remote processing unit operatively coupled with the computer 400, as explained above.

According to processing step 202, a model suitable for identifying the identifiable features is obtained.

In one embodiment, the model suitable for identifying the identifiable features is a Single Shot MultiBox Detector (SSD) model known to the skilled addressee. The skilled addressee will appreciate that various alternative embodiments may be provided for the model suitable for identifying the identifiable features. For instance and in accordance with another embodiment, the model suitable for identifying the identifiable features is a You Only Look Once (YOLO) model, known to the skilled addressee.

It will be also appreciated that the model suitable for identifying the identifiable features may be provided according to various embodiments.

In accordance with an embodiment, the model suitable for identifying the identifiable features is obtained from the memory unit 412 of the computer 400.

In accordance with another embodiment, the model suitable for identifying the identifiable features is provided by a user interacting with the computer 400.

In accordance with yet another embodiment, the model suitable for identifying the identifiable features is obtained from a remote processing unit operatively coupled with the computer 400 as explained above.

Still referring to FIG. 2 and according to processing step 204, an indication of identifiable entities is provided.

It will be appreciated that the indication of identifiable entities refers to elements that may be used to identify data such as morphometric patterns in imaging data, acoustic pattern in spectral data (albeit spectrogram), trending pattern in 1-D data.

For instance and in the case of patient data, the identifiable entities refer to elements that may be used to identify a patient.

In the context of imaging patient data, organs could be used to identify patient data, and accordingly said indication of identifiable entities could be a weak indication of organs' presence at the level of imaging patient data, organ bounding boxes on some imaging patient data, organ segmentation on some imaging patient data. Additional elements that may be used to identify patients are morphometry of the face either directly or indirectly obtained in the case of CT of the head for example, gait from videos, patient history and chronology of specific events, patient-specific morphology either from birth defects or surgically related.

It will be also appreciated that the indication of identifiable entities may be provided according to various embodiments.

In accordance with an embodiment, the indication of identifiable entities is obtained from the memory unit 412 of the computer 400.

In accordance with another embodiment, the indication of identifiable entities is provided by a user interacting with the computer 400.

In accordance with yet another embodiment, the indication of identifiable entities is obtained from a remote processing unit operatively coupled with the computer 400 as explained above.

Still referring to FIG. 2 and according to processing step 206, an identifier embedding is generated.

It will be appreciated that the identifier embedding is generated using the model suitable for identifying the identifiable features, the indication of identifiable entities and the data to be used for identifying the identifiable features.

In one embodiment, the identifier embedding is generated using the computer 400.

Now referring back to FIG. 1 and according to processing step 104, a task-specific embedding comprising task-specific features is generated.

It will be appreciated that the task-specific embedding comprising task-specific features may be generated according to various embodiments.

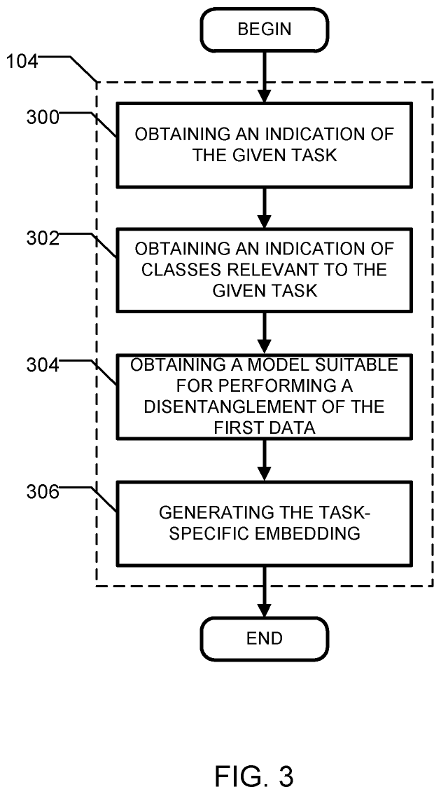

Now referring to FIG. 3, there is shown an embodiment for generating the task-specific embedding comprising task-specific features.

According to processing step 300, an indication of the given task is obtained.

As mentioned above, it will be appreciated that the indication of the given task may be of various types.

It will be also appreciated that the indication of the given task may be provided according to various embodiments.

In accordance with an embodiment, the indication of the given task is obtained from the memory unit 512 of the computer 500.

In accordance with another embodiment, the indication of the given task is provided by a user interacting with the computer 500.

In accordance with yet another embodiment, the indication of the given task is obtained from a remote processing unit operatively coupled with the computer 500 as explained above.

Still referring to FIG. 3 and according to processing step 302, an indication of classes relevant to the given task is provided.

It will be appreciated by the skilled addressee that the indication of classes relevant to the given task are at least binary, for instance responding, nonresponding—malignant/benign, or multi-classes, such as for instance disease progression, no progression, pseudo-progression.

It will be also appreciated that the indication of classes relevant to the given task may be provided according to various embodiments.

In accordance with an embodiment, the indication of classes relevant to the given task is obtained from the memory unit 412 of the computer 400.

In accordance with another embodiment, the indication of classes relevant to the given task is provided by a user interacting with the computer 400.

In accordance with yet another embodiment, the indication of classes relevant to the given task is obtained from a remote processing unit operatively coupled with the computer 400 as explained above.

Still referring to FIG. 3 and according to processing step 304, a model suitable for performing a disentanglement of the first data is provided.

In one embodiment, the model suitable for performing a disentanglement of the first data is the Adversarially Learned Mixture Model (AMM) disclosed herein.

It will be appreciated that alternative embodiments of the model suitable for performing a disentanglement of the data may be provided. In fact, it has been contemplated that any model capable of modeling complex data distribution may be used. It will be appreciated that the Generative Adversarial Network (GAN) has recently emerged as a powerful framework for modeling complex data distributions without having to approximate intractable likelihoods. As mentioned above and in a preferred embodiment an Adversarially Learned Mixture Model (AMM) is used, a generative model inferring both continuous and categorical latent variables to perform either unsupervised or semi-supervised clustering of data using a single adversarial objective, that explicitly model the dependence between continuous and categorical latent variables, and which eliminates discontinuities between categories in the latent space.

It will be also appreciated that the model suitable for performing a disentanglement of the first data may be provided according to various embodiments.

In accordance with an embodiment, the model suitable for performing a disentanglement of the first data is obtained from the memory unit 412 of the computer 400.

In accordance with another embodiment, the model suitable for performing a disentanglement of the first data is provided by a user interacting with the computer 400.

In accordance with yet another embodiment, the model suitable for performing a disentanglement of the first data is obtained from a remote processing unit operatively coupled with the computer 400 as explained above.

Still referring to FIG. 3 and according to processing step 306, a task-specific embedding is generated.

It will be appreciated that a task-specific embedding refers to one of a regression, a classification, a clustering, a multivariate querying, a density estimation, a dimension reduction and a testing and matching.

More precisely, the task-specific embedding is generated using the obtained model, the indication of classes relevant to the given task, the indication of the given task and the data. In another embodiment, the task-specific embedding is generated using the obtained model, the indication of classes relevant to the given task, the indication of the given task and the first data.

Such generation of the task-embedding can be performed, in a preferred embodiment, using the above-mentioned Adversarially Learned Mixture Model (AMM). In another embodiment, a generative model following “Learning disentangled representations with semi-supervised deep generative models—arXiv:1706.00400 [stat.ML]” may be used

Now referring back to FIG. 1 and according to processing step 106, the synthetically anonymized data for the given task is generated.

It will be appreciated that the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features. The generating further mixes the first sample and the second sample in a generative process to create the generated synthetically anonymized data.

In one embodiment, the first sampling from the data embedding which ensures that corresponding first sample originates away from a projection of the data and the first data in the identifier embedding is performed using a rejection sampling technique such as detailed in “Deep Learning for Sampling from Arbitrary Probability Distributions—arXiv:1801.04211”.

In another embodiment, the sampling process is performed using a Markov chain Monte Carlo (MCMC) sampling process such as detailed in “Improving Sampling from GenerativeAutoencoders with Markov Chains—OpenReview ryXZmzNeg—Antonia Creswell, Kai Arulkumaran, Anil Anthony Bharath 30 Oct. 2016 (modified: 12 Jan. 2017) ICLR 2017 conference submission”; accordingly, since, the generative model learns to map from the learned latent distribution, rather than the prior, a Markov chain Monte Carlo (MCMC) sampling process may be used to improve the quality of samples drawn from the generative model, especially when the learned latent distribution is far from the prior.

In yet a further embodiment, the sampling process includes Parallel Checkpointing Learners methods that ensure that although samples originates away from a projected a-priori known data in the identifiable embedding, the generative model is robust against adversarial samples, by rejecting samples that are likely to come from the unexplored regions conveying potentially high risk of irrelevance such as detailed in “Towards Safe Deep Learning: Unsupervised Defense Against Generic Adversarial Attacks—OpenReview Hyl6s40a-”.

In one embodiment, mixing samples originating from different embeddings is performed as disclosed in “conditional generative adversarial nets—arXiv:1411.1784”, in “Generative adversarial text to image synthesis—arXiv:1605.05396”, in “PixelBrush: Art generation from text with GANs—Jiale Zhi Stanford University” and in “RenderGAN: generating realistic labelled data—arXiv:1611.01331”.

Still referring to FIG. 1 and according to processing step 108, a check is performed in order to find out if the generated synthetically anonymized data is dissimilar to the first data to be anonymized for a given metric. It will be appreciated that processing step 108 is optional.

It will be appreciated that the given metric may be of various types as known to the skilled addressee.

In fact and in one embodiment, the checking that the generated synthetically anonymized data is dissimilar to the first data to be anonymized for a given metric, is performed following traditional image similarity measures as detailed in “Mitchell H. B. (2010) Image Similarity Measures. In: Image Fusion. Springer, Berlin, Heidelberg”, or following differential privacy as detailed in “Privacy-preserving generative deep neural networks support clinical data sharing—bioarxkiv:159756”, in “L. Sweeney, k-anonymity: A model for protecting privacy, Int. J. Uncertainty, Fuzziness (2002)”.

While it has been disclosed that the checking is performed following the generating step 106, it will be appreciated by the skilled addressee that in another alternative embodiment, the checking performed according to processing step 108 is integrated in the generating processing step disclosed in processing step 106 as detailed in “Generating differentially private datasets using GANs—OpenReview rJv4XWZA—, ICLR 2018”. In such embodiment, the checking step as disclosed in FIG. 1 is optional.

In such embodiment, the generating of the synthetically anonymized data for the given task comprises checking that the synthetically anonymized data is dissimilar to the first data to be anonymized for a given metric.

According to processing step 110, the generated synthetically anonymized data for the given task is provided. It will be appreciated that the generated synthetically anonymized data for the given task is provided if the checking is successful.

It will be appreciated that the generated synthetically anonymized data may be provided according to various embodiments.

In accordance with an embodiment, the generated synthetically anonymized data is stored in the memory unit 412 of the computer 400.

In accordance with another embodiment, the generated synthetically anonymized data is provided to a remote processing unit operatively coupled to the computer 400.

In another alternative embodiment, the generated synthetically anonymized data is displayed to a user interacting with the computer 400.

Still referring to FIG. 4, it will be appreciated that the application for generating synthetically anonymized data 416 comprises instructions for providing first data to be anonymized.

The application for generating synthetically anonymized data 416 further comprises instructions for providing a data embedding comprising data features, wherein data features enable a representation of corresponding data wherein the data is representative of the first data.

The application for generating synthetically anonymized data 416 further comprises instructions for providing an identifier embedding comprising identifiable features. It will be appreciated that the identifiable features enable an identification of the first data.

The application for generating synthetically anonymized data 416 further comprises instructions for providing a task-specific embedding comprising task specific features suitable for the task. It will be appreciated that the task specific features enable a disentanglement of different classes relevant to the given task.

The application for generating synthetically anonymized data for the given task further comprises instructions for generating synthetically anonymized data for the given task, wherein the generating comprises a generative process using samples comprising a first sampling from the data embedding which ensures that a corresponding first sample originates away from a projected data and the first data in the identifiable embedding and a second sampling from the task-specific embedding which ensures that a corresponding second sample originates close to the task-specific features and wherein the generating further mixes the first sample and the second sample in a generative process to create the generated synthetically anonymized data.

The application for generating synthetically anonymized data for the given task further comprises instructions for checking that the synthetically anonymized data is dissimilar to the first data to be anonymized for a given metric.

The application for generating synthetically anonymized data for the given task further comprises instructions for providing the generated synthetically anonymized data for the given task if said checking is successful.

A non-transitory computer readable storage medium is disclosed for storing computer-executable instructions which, when executed, cause a computer to perform a method for generating synthetically anonymized data for a given task, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data; providing a task-specific embedding comprising task-specific features suitable for said task, wherein said task-specific features enables a disentanglement of different classes relevant to the given task; generating synthetically anonymized data for the given task, wherein the generating comprises a generative process using samples comprising a first sampling from the data embedding which ensures that a corresponding first sample originates away from a projected data and the first data in the identifiable embedding and a second sampling from the task-specific embedding which ensures that a corresponding second sample originates close to the task-specific features and wherein the generating further mixes the first sample and the second sample in a generative process to create the generated synthetically anonymized data; checking that the synthetically anonymized data is dissimilar to the first data to be anonymized for a given metric and providing the generated synthetically anonymized data for the given task if the checking is successful.

It will be appreciated that the method disclosed herein is of great advantage for various reasons.

In fact, a first advantage of the method disclosed is that it provides privacy by-design for an anonymization process, while ensuring that the anonymized data is relevant for further research pertaining to a given task and to be representative of the general “look'n'feel” of the original data.

A second advantage of the method disclosed herein is that it enables the sharing of patient data in an open innovation environment, while ensuring patient privacy and control over the specific characteristics of the anonymized data (representative of all patient or sub-population thereof, representative globally of a task or sub-classes thereof).

A third advantage of the method disclosed herein is that it provides ways to anonymize data without a-priori on what aspects of the data may convey such privacy risk(s); accordingly as such risk evolve, the method disclosed herein may adapt and benefit from further research and development in the field of data privacy.

Adversarially Learned Mixture Model (AMM)

It will be appreciated that the Adversarially Learned Mixture Model (AMM) is disclosed herein below. This model may be used advantageously in the method disclosed herein as mentioned previously.

It is known to the skilled addressee that the ALI and BiGAN models are trained by matching two joint distributions of images x∈ [symbol] ^{D} and their latent code z∈ [symbol] ^{L}. The two distributions to be matched are the inference distribution q(x,z) and the synthesis distribution p(x,z), wherein,

q(x,z)=q(x)q(z|x), Equation (1)

p(x,z)=p(z)p(x|z). Equation (2)

Samples of q(x) are drawn from the training data and samples of p(z) are drawn from a prior distribution, usually [symbol] (0,1). Samples from q(z|x) and p(x|z) are drawn from neural networks that are optimized during training. Dumoulin et al. (See “Adversarially learned inference”. in International Conference on Learning Representation (2016)) show that sampling from q(z|x)= [symbol] (μ(x),σ²(x)I) is possible by employing the reparametrization trick (See Kingma & Welling, “Auto-encoding variational Bayes”, in International Conference on Learning Representation (2013)), i.e. computing:

z=μ(x)+σ(x)⊙ϵ,ϵ˜ [symbol] (0,I), Equation (3)

wherein ⊙ is the element wise vector multiplication.

A conditional variant of ALI has also been explored by Dumoulin et al. (2016) wherein an observed class-conditional categorical variable y has been introduced. The joint factorization of each distribution to be matched are:

q(x,y,z)=q(x,y)q(z|y,x), Equation (4)

p(x,y,z)=p(y)p(z)q(x|y,z). Equation (5)

It will be appreciated that samples of q(x,y) are drawn from the data, samples of p(z) are drawn from a continuous prior on z, and samples of p(y) are drawn from a categorical prior on y, both of which are marginally independent. It will be further appreciated that samples from q(z|y,x) and p(x|y,z) are drawn from neural networks that are optimized during training.

In the following, graphical models are presented for q(x,y,z) and p(x,y,z) that build off of conditional ALI. Where conditional ALI requires the full observation of categorical variables, the models presented account for both unobserved and partially observed categorical variables.

Adversarially Learned Mixture Model

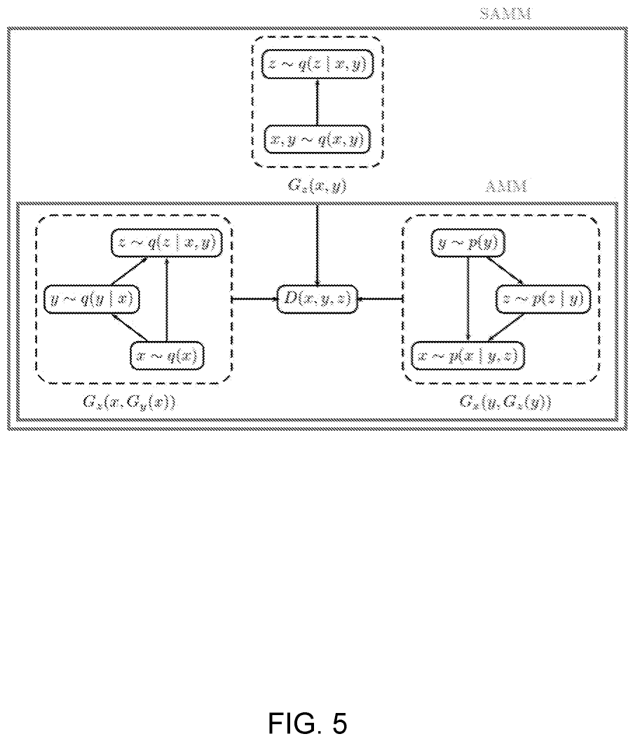

It will be appreciated that the Adversarially Learned Mixture Model (AMM) disclosed herein and illustrated in FIG. 5 is an adversarial generative model for deep unsupervised clustering of data.

Like conditional ALI, a categorical variable is introduced to model the labels.

However, the unsupervised setting requires a different factorization of the inference distribution in order to enable inference of the categorical variable y, namely:

q₁(x,y,z)=q(x)q(y|x)q(z|x,y), Equation (6)

or

q₂(x,y,z)=q(x)q(z|x)q(y|x,z). Equation (7)

Samples of q(x) are drawn from the training data, and samples from q(y|x), q(z|x, y) or q(z|x), q(y|x, z) are generated by neural networks. It will be appreciated that the reparametrization trick is not directly applicable to discrete variables and multiple methodologies have been introduced to approximate categorical samples (See Jang et al. “Categorical reparametrization with Gumbel-softmax”. arXiv preprint arXiv:1611.01144, 2016; Maddison et al. “The concrete Distribution: A Continuous Relaxation of Discrete Random Variables.” in International Conference on learning representations, 2017). It will be appreciated that in this embodiment Kendall & Gal (See “What uncertainties do we need in Bayesian deep learning for computer vision? In Advances in Neural Information Processing Systems 30, pp. 5580-5590 (2017)) is followed and a sample is performed from q(y|x) by computing:

h_{y}(x)=μ(x)+σ_{y}(x)⊙ϵ,ϵ˜ [symbol] (0,I), Equation (8)

y(x)=softmax(h_{y}(x)). Equation (9)

It is then possible to sample from q(z|x, y), by computing:

z(x,h_{y}(x))=μ_{z}(x,h_{y}(x))+σ_{z}(x,h_{y}(x))⊙ϵ,ϵ˜ [symbol] (0,I). Equation (10)

A similar sampling strategy may be used to sample from q(y|x, z) in Equation (7).

The factorization of the synthesis distribution p(x,y,z) also differs from conditional ALI:

p(x,y,z)=p(y)p(z|y)p(x|y,z). Equation (11)

It will be appreciated that the product p(y)p(z|y) may be conveniently given by a mixture model. Samples from p(y) are drawn from a multinomial prior, and samples from p(z|y) are drawn from a continuous prior, for example, [symbol] (μ_{y=k},1). Samples from p(z|y) may alternatively be generated by a neural network by again employing the reparameterization trick, namely:

z(y)=μ(y)+σ(y)⊙ϵ,ϵ˜ [symbol] (0,I). Equation (12)

This approach effectively learns the parameters of [symbol] (μ_{y=k}, σ_{y=k}).

Adversarial Value Function

Dumoulin et al. (2016) is followed and the value function that describes the unsupervised game between the discriminator D and the generator G is defined as:

min_{G} max_{D} V(D,G)= [symbol] _{q(x)}[log(D(x,G_{y}(x),G_{z}(x,G_{y}(x))))]+ [symbol] _{p(y,z)}[log(1−D(Gₓ(y,G_{z}(y)),y,G_{z}(y))]=∫∫∫q(x)q(y|x)q(z|x,y)log(D(x,y,z))dx dy dz+∫∫∫p(y)p(z|y)p(x|y,z)log(1−D(x,y,z))dx dy dz Equation (13)

It will be appreciated that there are four generators in total: two for the encoder G_{y}(x) and G_{z}(x, G_{y}(x)), which map the data samples to the latent space, and two for the decoder G_{z}(y) and Gₓ(y, G_{z}(y)), which map samples from the prior to the input space. G_{z}(y) can either be a learned function, or be specified by a known prior. A detailed description of the optimization procedure is detailed herein below.

Algorithm 1 AMM training procedure using distributions (6) and (11).

| | |
|---|---|
| θ [symbol] , θ [symbol] ( [symbol] , [symbol] (x)),θ [symbol] ( [symbol] ),θ [symbol] ( [symbol] , [symbol] ( [symbol] )),θ_{D} | [symbol] Initialize AMM parameters |
| while do not done do | |
| x⁽¹⁾ [symbol] x^{(M)} ~ q(x) | [symbol] Sample from data and priors |
| y⁽¹⁾ [symbol] y^{(M)} ~ p(y) | |
| z^{(j)} ~ p(z\|y = y^{(j)}), j = 1, . . . , M | |
| [symbol] ^{(j)} ~ p(x\|y = y^{(j)}, z = z^{(j)}), j = 1, . . . , M | [symbol] Sample from conditionals |
| [symbol] ⁽ⁱ⁾ ~ q(y\|x = x⁽ⁱ⁾), i = 1, . . . , M | |
| [symbol] ⁽ⁱ⁾ ~ q(z\|x = x⁽ⁱ⁾, y = [symbol] ⁽ⁱ⁾), i = 1, . . . , M | |
| ρ_{q}⁽ⁱ⁾ ← D(x⁽ⁱ⁾, [symbol] ⁽ⁱ⁾, [symbol] ⁽ⁱ⁾), i = 1, . . . , M | [symbol] Compute discriminator predictions |
| ρₚ^{(j)} ← D( [symbol] ^{(j)},y^{(j)},z^{(j)}), j = 1, . . . , M | |
| ${ℒ}_{D} ← - \frac{1}{M} {Σ}_{i = m}^{M} log ( {ρ}_{q}^{( i )} ) - \frac{1}{M} {Σ}_{j = 1}^{M} log ( 1 - {ρ}_{p}^{( j )} )$ | [symbol] Compute discriminator losses |
| $ℒ \text{?} ( \text{?} , \text{?} ( \text{?} ) ) = ℒ \text{?} ( \text{?} ) ← - \frac{1}{M} {Σ}_{i = 1}^{M} log ( {ρ}_{p}^{( i )} )$ | [symbol] Compute x generator losses |
| $ℒ \text{?} ( \text{?} ) = ℒ \text{?} ( \text{?} , \text{?} ( \text{?} ) ) ← - \frac{1}{M} {Σ}_{i = 1}^{M} log ( 1 - {ρ}_{q}^{( i )} )$ | [symbol] Compute y and z generator loss |
| θ_{D} ← θ_{D} − ∇ [symbol] [symbol] | [symbol] Update discriminator parameters |
| [symbol] [symbol] [symbol] [symbol] ← [symbol] ( [symbol] , [symbol] [symbol] ) − ∇ [symbol] [symbol] [symbol] [symbol] [symbol] | [symbol] Update generator parameters |
| [symbol] [symbol] [symbol] ← [symbol] [symbol] − ∇ [symbol] [symbol] [symbol] [symbol] [symbol] | |
| [symbol] [symbol] [symbol] ← [symbol] [symbol] − ∇ [symbol] [symbol] [symbol] [symbol] [symbol] | |
| [symbol] [symbol] [symbol] [symbol] ← [symbol] ( [symbol] , [symbol] [symbol] ) − ∇ [symbol] [symbol] [symbol] [symbol] [symbol] | |

[symbol]

indicates data missing or illegible when filed

Semi-Supervised Adversarially Learned Mixture Model

The Semi-Supervised Adversarially Learned Mixture Model (SAMM) is an adversarial generative model for supervised or semi-supervised clustering and classification of data. The objective for training the Semi-Supervised Adversarially Learned Mixture Model involves two adversarial games to match pairs of joint distributions. The supervised game matches inference distribution (4) to synthesis distribution (11) and is described by the following value function:

min_{G} max_{D}V(D,G)= [symbol] _{q(x,y)}[log(D(x,y,G_{z}(x,y)))]+ [symbol] _{p(y,z)}[log(1−D(Gₓ(y,G_{z}(y)),y,G_{z}(y)))]=∫∫∫q(x,y)q(z|x,y)log(D(x,y,z))dx dy dz+∫∫∫p(y)p(z|y)p(x|y,z)log(1−D(x,y,z))dx dy dz.

### CLAUSES

Clause 1. A method for generating synthetically anonymized data for a given task, the method comprising:
providing first data to be anonymized;
providing a data embedding comprising data features, wherein data features enable a representation of corresponding data, and wherein the data is representative of the first data;
providing an identifier embedding comprising identifiable features, wherein the identifiable features enable an identification of the data and the first data;
providing a task-specific embedding comprising task-specific features suitable for said task, wherein said task-specific features enable a disentanglement of different classes relevant to the given task;
generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and
providing the generated synthetically anonymized data for the given task.

Clause 2. The method as claimed in clause 1, wherein the generating of the synthetically anonymized data for the given task comprises checking that the synthetically anonymized data is dissimilar to the first data to be anonymized for a given metric; further wherein the generated synthetically anonymized data for the given task is provided if said checking is successful.

Clause 3. The method as claimed in any one of clauses 1 to 2, wherein the first data comprises patient data.

Clause 4. The method as claimed in any one of clauses 1 to 3, wherein the providing of the task-specific embedding comprising task specific features suitable for said task comprises:
obtaining an indication of the given task;
obtaining an indication of classes relevant to the given task;
obtaining a model suitable for performing a disentanglement of the data for the given task; and
generating the task-specific embedding using the obtained model, the indication of classes relevant to the given task, the indication of the given task and the data.

Clause 5. The method as claimed in any one of clauses 1 to 4, wherein the providing of the identifier embedding comprising identifiable features comprises:
obtaining data used for identifying the identifiable features;
obtaining a model suitable for identifying the identifiable features in said data;
obtaining an indication of identifiable entities; and
generating the identifier embedding using the model suitable for identifying the identifiable features, the indication of identifiable entities and the data to be used for identifying the identifiable features.

Clause 6. The method as claimed in clause 5, wherein the data comprises the data used for identifying the identifiable features.

Clause 7. The method as claimed in clause 5, wherein the model suitable for identifying the identifiable features in said data comprises a Single Shot MultiBox Detector (SSD) model.

Clause 8. The method as claimed in clause 4, wherein the model suitable for performing a disentanglement of the data for the given task comprises one of an Adversarially Learned Mixture Model (AMM) in one of a supervised, semi supervised or unsupervised training.

Clause 9. The method as claimed in clause 4, wherein the indication of identifiable entities comprises one of a number of classes and an indication of a class corresponding to at least one of said data.

Clause 10. The method as claimed in clause 5, wherein the indication of identifiable entities comprises at least one box locating at least one corresponding identifiable entity.

Clause 11. A non-transitory computer readable storage medium for storing computer-executable instructions which, when executed, cause a computer to perform a method for generating synthetically anonymized data for a given task, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data and the first data; providing a task-specific embedding comprising task-specific features suitable for said task, wherein said task-specific features enables a disentanglement of different classes relevant to the given task; generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and providing the generated synthetically anonymized data for the given task.

Clause 12. A computer comprising:
a central processing unit;
a display device;
a communication unit;
a memory unit comprising an application for generating synthetically anonymized data for a given task, the application comprising:
   instructions for providing first data to be anonymized;
instructions for providing a data embedding comprising data features, wherein data features enable a representation of corresponding data, and wherein the data is representative of the first data;
   instructions for providing an identifier embedding comprising identifiable features, wherein the identifiable features enable an identification of the data and the first data;
   instructions for providing a task-specific embedding comprising task-specific features suitable for said task, wherein said task-specific features enables a disentanglement of different classes relevant to the given task;
   instructions for generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and
   instructions for providing the generated synthetically anonymized data for the given task.

Although the above description relates to a specific preferred embodiment as presently contemplated by the inventor, it will be understood that the invention in its broad aspect includes functional equivalents of the elements described herein.

1. A method for generating synthetically anonymized data for a given task, the method comprising:
providing first data to be anonymized;
providing a data embedding comprising data features, wherein data features enable a representation of corresponding data, and wherein the data is representative of the first data;
providing an identifier embedding comprising identifiable features, wherein the identifiable features enable an identification of the data and the first data;
providing a task-specific embedding comprising task-specific features suitable for said task, wherein said task-specific features enable a disentanglement of different classes relevant to the given task;
generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and
providing the generated synthetically anonymized data for the given task.

2. The method as claimed in claim 1, wherein the generating of the synthetically anonymized data for the given task comprises checking that the synthetically anonymized data is dissimilar to the first data to be anonymized for a given metric; further wherein the generated synthetically anonymized data for the given task is provided if said checking is successful.

3. The method as claimed in any one of claims 1 to 2, wherein the first data comprises patient data.

4. The method as claimed in any one of claims 1 to 3, wherein the providing of the task-specific embedding comprising task specific features suitable for said task comprises:
obtaining an indication of the given task;
obtaining an indication of classes relevant to the given task;
obtaining a model suitable for performing a disentanglement of the data for the given task; and
generating the task-specific embedding using the obtained model, the indication of classes relevant to the given task, the indication of the given task and the data.

5. The method as claimed in any one of claims 1 to 4, wherein the providing of the identifier embedding comprising identifiable features comprises:
obtaining data used for identifying the identifiable features;
obtaining a model suitable for identifying the identifiable features in said data;
obtaining an indication of identifiable entities; and
generating the identifier embedding using the model suitable for identifying the identifiable features, the indication of identifiable entities and the data to be used for identifying the identifiable features.

6. The method as claimed in claim 5, wherein the data comprises the data used for identifying the identifiable features.

7. The method as claimed in claim 5, wherein the model suitable for identifying the identifiable features in said data comprises a Single Shot MultiBox Detector (SSD) model.

8. The method as claimed in claim 4, wherein the model suitable for performing a disentanglement of the data for the given task comprises one of an Adversarially Learned Mixture Model (AMM) in one of a supervised, semi supervised or unsupervised training.

9. The method as claimed in claim 4, wherein the indication of identifiable entities comprises one of a number of classes and an indication of a class corresponding to at least one of said data.

10. The method as claimed in claim 5, wherein the indication of identifiable entities comprises at least one box locating at least one corresponding identifiable entity.

11. A non-transitory computer readable storage medium for storing computer-executable instructions which, when executed, cause a computer to perform a method for generating synthetically anonymized data for a given task, the method comprising providing first data to be anonymized; providing a data embedding comprising data features, wherein data features enable a representation of corresponding data, and wherein the data is representative of the first data; providing an identifier embedding comprising identifiable features, wherein the identifiable features enable an identification of the data and the first data; providing a task-specific embedding comprising task-specific features suitable for said task, wherein said task-specific features enables a disentanglement of different classes relevant to the given task; generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and providing the generated synthetically anonymized data for the given task.

12. A computer comprising:
a central processing unit;
a display device;
a communication unit;
a memory unit comprising an application for generating synthetically anonymized data for a given task, the application comprising:
instructions for providing first data to be anonymized;
instructions for providing a data embedding comprising data features, wherein data features enable a representation of corresponding data, and wherein the data is representative of the first data;
instructions for providing an identifier embedding comprising identifiable features, wherein the identifiable features enable an identification of the data and the first data;
instructions for providing a task-specific embedding comprising task-specific features suitable for said task, wherein said task-specific features enables a disentanglement of different classes relevant to the given task;
instructions for generating synthetically anonymized data for the given task, wherein the generating comprises a generative process using samples comprising a first sampling from the data embedding which ensures that a corresponding first sample originates away from a projection of the data and the first data in the identifier embedding and a second sampling from the task-specific embedding which ensures that a corresponding second sample originates close to the task-specific features and wherein the generating further mixes the first sample and the second sample in a generative process to create the generated synthetically anonymized data; and
instructions for providing the generated synthetically anonymized data for the given task.
